(19) United States
(12) Patent Application Publication
(10) Pub. No.: US 2005/0069871 A1
(43) Pub. Date: Mar. 31, 2005

(54) Polypeptide-human zinc finger protein fpm315-17 and the polynucleotide encoding it

(76) Inventors: Yumin Mao, Shanghai (CN); Yi Xie, Shanghai (CN)

Correspondence Address: CROWELL & MORING LLP;INTELLECTUAL PROPERTY GROUP, P.O. BOX 14300, WASHINGTON, DC, 20044-4300, US

(21) Appl. No.: 10/311,632

(22) PCT Filed: Jun. 18, 2001
(86) PCT No.: PCT/CN01/01000

(30) Foreign Application Priority Data
Jun. 19, 2000 (CN) 00116574.7

(51) Int. Cl. C12Q 1/68; C07H 21/04; C12N 9/64

(52) U.S. Cl. 435/6; 435/69.1; 435/226; 435/320.1; 435/325; 536/23.2

(57) ABSTRACT

A new polypeptide, human zinc finger protein FPM315-17, the polynucleotide encoding it and a method for producing the polypeptide by DNA recombinant technology. The present invention further discloses a method for using the polypeptide or polynucleotide for treating various disorders, e.g., various tumors, nervous system disorders, hemopathy, developmental disorders, and HIV infections. The present invention also discloses an antagonist of the polypeptide and its therapeutic application.

### FIELD OF THE INVENTION

The invention relates to the field of biotechnology. In particular, the invention relates to a novel polypeptide, human zinc finger protein FPM315-17, and a polynucleotide sequence encoding said polypeptide. The invention also relates to the method for the preparation and use of said polynucleotide and polypeptide.

### TECHNICAL BACKGROUND

C2H2 zinc finger configuration is formed by the interaction between two pairs of conserved Cys and His residues and a zinc ion (A. D. Frankel, J. M. Berg, C. O. Pabo, 1987). This kind of zinc finger configuration can be found in many transcriptional factors, which have great influence on the growth, differentiation and development of the cell (T. El-Baradi, T. Pieler, 1991). The zinc finger protein FPM315 is a member of C2H2-type zinc finger proteins.

Zinc finger protein FPM315 has 683 amino acid residues. There is an in frame termination codon, ATG, before its transcription start site, and a poly A signal (AATAAA) at the site of 3232-3237 in its sequence. There are nine C2H2 zinc finger domains in the C-terminal of this protein. These domains can be compartmentalized into 3 units. The first unit (from site 380 to 400) has one C2H2 domain, the second unit (from site 436-540) has four C2H2 domains, and the third unit (from site 577 to 681) also has four C2H2 domains. Cys and His residues are highly conserved in the zinc finger domain. There is a consensus sequence (C-X2-C-X3-F-X5-L-X2-H-X3-H) in each zinc finger domain. The second and the third units are connected by a highly conserved H/C sequence with the consensus sequence of T-G-E-K-K-P-Y-X (R. Schuh, W. Aicher et al., 1986).

There is a Leu-rich domain, called LeR, in the N-terminal of the zinc finger protein FPM315 (G. Pengue, V. Calabro et al., 1994), which is highly conserved in a lot of zinc finger proteins. LeR is rich in Leu and Glu, and can form a negatively charged domain that can be found in normal transcription factors. There are also two a-helixes, indicating that it may mediate the interaction between this protein and the components of the transcription complex.

There is a highly conserved KRAB region in almost one third of the C2H2 type zinc finger proteins (E. J., Bellefroid, D. A. Poncelet et al., 1991; M. Rosati, M. Marino et al., 1991), including KRAB-A and KRAB-B. In FPM315 there is only one KRAB-A region, which acts as a transcription repression region. (G. Pengue, V. Calabro et al., 1994; R. Witzgall, E. Oleary et al., 1993).

The mRNA of the zinc finger protein FPM315 is expressed in many tissues of infant and adult, including human tumor cell lines and lymphocyte cell lines. The widespread expression of this gene indicates that FPM315 is very important in fundamental cell activities like the synthesis of the proteins and the metabolism of the cells (P. J. Mitchell, R. Tijan, 1989).

The polypeptide of the instant invention has 58 percent identity and 75 percent similarity with zinc finger protein FPM315. It has the characteristic consensus sequence of the zinc finger protein family. It is determined to be a new C2H2-type zinc finger protein with similar biological functions of zinc finger protein FPM315, designated human zinc finger protein FPM315-17.

Human zinc finger protein FPM315-17 plays an essential role in the regulation of important biological functions such as cell division and embryogenesis. Thus, the identification and isolation of human zinc finger protein FPM315-17, especially of its amino acid sequence is always desired in this filed. The isolation of this novel human zinc finger protein FPM315-17 builds the basis for research of the protein function under normal and clinical conditions, disease diagnosis and drug development.

### DESCRIPTION OF THE INVENTION

One objective of the invention is to provide an isolated novel polypeptide, i.e., a human zinc finger protein FPM315-17, and fragments, analogues and derivatives thereof.

Another objective of the invention is to provide a polynucleotide encoding said polypeptide.

Another objective of the invention is to provide a recombinant vector containing a polynucleotide encoding a human zinc finger protein FPM315-17.

Another objective of the invention is to provide a genetically engineered host cell containing a polynucleotide encoding a human zinc finger protein FPM315-17.

Another objective of the invention is to provide a method for producing a human zinc finger protein FPM315-17.

Another objective of the invention is to provide an antibody against a human zinc finger protein FPM315-17 of the invention.

Another objective of the invention is to provide mimetics, antagonists, agonists, and inhibitors for the polypeptide of the human zinc finger protein FPM315-17.

Another objective of the invention is to provide a method for the diagnosis and treatment of the diseases associated with an abnormality of human zinc finger protein FPM315-17.

The present invention relates to an isolated polypeptide, which is originated from human, and comprises a polypeptide having the amino acid sequence of SEQ ID NO: 2, or its conservative mutants, or its active fragments, or its active derivatives or its analogues. Preferably, the polypeptide has the amino acid sequence of SEQ ID NO: 2.

The present invention also relates to an isolated polynucleotide, comprising a nucleotide sequence or its variant selected from the group consisting of (a) the polynucleotide encoding a polypeptide comprising the amino acid sequence of SEQ ID NO: 2; (b) a polynucleotide complementary to the polynucleotide (a); and (c) a polynucleotide shares at least 70% homology to the polynucleotide (a) or (b). More preferably, said nucleotide sequence is selected from the group consisting of (a) the sequence of position 224-685 in SEQ ID NO: 1; and (b) the sequence of position 1-1284 in SEQ ID NO: 1.

The invention also includes: a vector containing the polynucleotides of the invention, especially an expression vector; a host cell genetically engineered with the vector, and the host cell can be produced via transformation, transduction or transfection; a method for the production of the inventive polypeptide through the process of host cell cultivation and expession product harvest.

The invention also involves an antibody which specifically binds to the invention polypeptide.

The invention also includes a method for selecting compounds which could mimic, activate, antagonize, or repress the activity of the human zinc finger protein FPM315-17, and the compounds obtained by the method.

The invention also includes a method for an in vitro assay of diseases or disease susceptibility related with the abnormal expression of human zinc finger protein FPM315-17. The method involves mutation detection of the said polypeptide or its encoding polynucleotide sequence, or the quantitative detection or biological activity assay of the polypeptide in biological samples.

The present invention also includes a pharmaceutical composition which includes the polypeptide, its mimetic, its agonist, its antagonist, or its repressor, and a pharmaceutically acceptable carrier.

The invention also includes application of said invented polypeptide and/or its polynucleotide for drug development to treat cancers, developmental diseases, immune diseases, or other diseases caused by abnormal expression of human zinc finger protein FPM315-17.

Other aspects of the invention are apparent to the skilled in the art in view of the instant disclosure.

The terms used in this specification and claims have the following meanings, unless specifically defined otherwise.

“Nucleotide sequence” refers to oligonucleotide, nucleotide, or polynucleotide and parts of polynucleotide. It includes genomic or synthetic DNA or RNA, which could be single-stranded or double-stranded, and could represent the sense strand or the antisense strand. Similarly, the term “amino acid sequence” refers to oligopeptide, peptide, polypeptide, or protein sequence and parts of proteins. When the “amino acid sequence” is related to the sequence of a natural protein, the amino acid sequence of the “peptide” or “protein” is not limited to be identical to the sequence of that natural protein.

“Variant” of a protein or polynucleotide refers to the amino acid sequence with one or several amino acid changed, or its encoding polynucleotide sequence with one or several nucleotides changed, respectively. Such changes include deletion, insertion, or substitution of amino acids in the amino acid sequence, or of nucleotides in the polynucleotide sequence. In the context of peptide variant, these changes could be conservative and the substituted amino acid has similar structure or chemical characteristics as the original one, just as the substitution of Ile with Leu. Changes also could be not conservative, just as the substitution of Ala with Trp.

“Deletion” refers to the deletion of one or several amino acids in the amino acid sequence, or of one or several nucleotides in the nucleotide sequence.

“Insertion” or “addition” refers to the addition of one or several amino acids in the amino acid sequence, or of one or several nucleotides in the nucleotide sequence, comparing to the natural molecule. “Substitution” refers to the change of one or several amino acids, or of one or several nucleotides, into different ones without changing the length of the sequence.

“Biological activity” refers to the structural, regulational or biochemical functions of a protein. Similarly, the term “immunological activity” refers to the ability of a natural, recombinant, or synthetic protein to induce a specific immunological reaction, or of binding to specific antibody in an appropriate animal or cell.

“Agonist” refers to a molecule which could up-regulate the activity of human zinc finger protein FPM315-17 by binding or changing it. Agonists may be proteins, nucleotides, carbohydrates or any other molecules.

“Antagonist” or “repressor” refers to the kind of molecule which could repress or downregulate the biological activity or immune activity of human zinc finger protein FPM315-17. Antagonists or repressores may be proteins, nucleotides, carbohydrates or any other molecules.

“Regulation” refers to functional changes of a protein, including increase or decrease of the protein activity, changes in binding specifity, changes of any other biological characteristics, functional or immunological characteristics.

“Substantially pure” refers to a condition of purity where the molecule at issue exists without any other naturally related proteins, lipids, saccharides, or other substances. Those of ordinary skills can purify human zinc finger protein FPM315-17 by standard protein purification techniques. Substantially pure human zinc finger protein FPM315-17 produces a single main band in denaturing polyacrylamide gel. The purity of human zinc finger protein FPM315-17 can be analyzed by amino acid sequence analysis.

“Complementary” or “complementation” refers to the natural conjugation of polynucleotides by base pairing under appropriate ion and temperature concentrations. For instance, the sequence 5′-C-T-G-A-3′ could bind to its complementary sequence 3′-G-A-C-T-5′. The complementation between two single-stranded nucleic acid molecules could be partial or complete. Complementary degree between two single strands has obvious influence on the efficiency of hybrid formation and the strength of the hybrid formed.

“Homology” refers to the complementary degree. Homology may be partial or complete. “Partial homology” refers to a partially complementary sequence compared to a target nucleotide, and the sequence could at least partially inhibit the hybridization between a completely complementary sequence and the target nucleotide. Inhibition of the hybridization could be assayed by hybridization (e.g., Southern blot or Northern blot) under a lower stringency condition. Substantially complementary sequence or hybrid probe could compete with the completely complementary sequence and inhibit its hybridization with the target sequence under a lower stringency condition. This effect does not mean that nonspecific binding is allowed under a lower stringency condition, because specific or selective reaction is still required for hybridization under a lower stringency condition.

“Percent identity” refers to the percentage of sequence identity or similarity when two or several amino acid or nucleotide sequences are compared. Percent identity could be determined by computation method such as MEGALIGN program (Lasergene software package, DNASTAR, Inc., Madison Wis.). MEGALIGN program can compare two or several sequences with different of methods such as the Cluster method (Higgins, D. G. and P. M. Sharp (1988) Gene 73:237-244). The Cluster method examines the distance between all pairs and arrange the sequences into clusters. Then the clusters are partitioned by pair or group. The percent identity between two amino acid sequences such as sequence A and B can be calculated by the following equation:

$$\frac{Number of paired residues between sequence A and B}{\begin{matrix}Residue number of sequence A - number of spacing residues \\ in sequence A - number of spacing residue in sequence B\end{matrix}} \times 100$$

Percent identity between nucleotide sequences can be determined by Cluster method or other well-known methods in this field such as the Jotun Hein method (Hein J., (1990) Methods in emzumology 183:625-645)

“Similarity” refers to the identical degree or conservative substitution degree of amino acid residues in corresponding sites of the amino acid sequences compared to each other. Amino acids for conservative substitution are: negative charged amino acids including Asp and Glu; positive charged amino acids including Leu, Ile and Val; Gly and Ala; Asn and Gln; Ser and Thr; Phe and Tyr.

“Antisense” refers to the nucleotide sequences complementary to a specific DNA or RNA sequence. “Antisense strand” refers to the nucleotide strand complementary to the “sense strand.”

“Derivative” refers to the inventive protein or a chemically modified nucleotide encoding it. This kind of modified chemical can be derived from replacement of the hydrogen atom with alkyl, acyl, or amino. The nucleotide derivative can encode peptide retaining the major biological characteristics of the natural molecule.

“Antibody” refers to the intact antibody or its fragments such as Fa, F(ab′)₂ and Fv, and it can specifically bind to epitope(s) of human zinc finger protein FPM315-17.

“Humanized antibody” refers to the antibody which has its amino acid sequence in non-antigen binding region replaced to mimic human antibody and still retain the original binding activity.

The term “isolated” refers to the removal of a material out of its original environment (for instance, if the substance is naturally produced, its original environment refers to its natural environment). For example, a naturally produced polynucleotide or a peptide in a living organism means it is not “isolated.” While the separation of the polynucleotide or a peptide from its coexisting materials in natural system means it is “isolated.” This polynucleotide may be a part of a vector. This polynucleotide or peptide may also be part of a compound. Since the vector or compound is not part of its natural environment, the polynucleotide or peptide is “isolated.”As used herein, the term “isolated” refers to a substance which has been isolated from the original environment. For naturally occurring substance, the original environment is the natural environment. For example, the polynucleotide and polypeptide in a naturally occurring state in the viable cells are not isolated or purified. However, if the same polynucleotide and polypeptide have been isolated from other components naturally accompanying them, they are isolated or purified.

As used herein, “isolated human zinc finger protein FPM315-17,” means that human zinc finger protein FPM315-17, does not essentially contain other proteins, lipids, carbohydrate or any other substances associated therewith in nature. The skilled in the art can purify human zinc finger protein FPM315-17, by standard protein purification techniques. The purified polypeptide forms a single main band on a non-reductive PAGE gel. The purity of human zinc finger protein FPM315-17 can be analyzed by amino acid sequence analysis.

The invention provides a novel polypeptide—human zinc finger protein FPM315-17, which comprises the amino acid sequence shown in SEQ ID NO: 2. The polypeptide of the invention may be a recombinant polypeptide, natural polypeptide, or synthetic polypeptide, preferably a recombinant polypeptide. The polypeptide of the invention may be a purified natural product or a chemically synthetic product. Alternatively, it may be produced from prokaryotic or eukaryotic hosts, such as bacterial, yeast, higher plant, insect, and mammal cells, using recombinant techniques. Depending on the host used in the protocol of recombinant production, the polypeptide of the invention may be glycosylated or non-glycosylated. The polypeptide of the invention may or may not comprise the starting Met residue.

The invention further comprises fragments, derivatives and analogues of human zinc finger protein FPM315-17. As used in the invention, the terms “fragment,” “derivative” and “analogue” mean the polypeptide that essentially retains the same biological functions or activity of human zinc finger protein FPM315-17 of the invention. The fragment, derivative or analogue of the polypeptide of the invention may be (i) one in which one or more of the amino acid residues are substituted with a conserved or non-conserved amino acid residue (preferably a conserved amino acid residue) and such substituted amino acid residue may or may not be one encoded by the genetic code; or (ii) one in which one or more of the amino acid residues are substituted with other residues, including a substituent group; or (iii) one in which the mature polypeptide is fused with another compound, such as a compound to increase the half-life of the polypeptide (for example, polyethylene glycol); or (iv) one in which additional amino acids are fused to the mature polypeptide, such as a leader or secretory sequence or a sequence which is employed for purification of the mature polypeptide or a proprotein sequence. Such fragments, derivatives and analogs are deemed to be within the scope of the skilled in the art from the teachings herein.

The invention provides an isolated nucleic acid or polynucleotide which comprises the polynucleotide encoding an amino acid sequence of SEQ ID NO: 2. The polynucleotide sequence of the invention includes the nucleotide sequence of SEQ ID NO: 1. The polynucleotide of the invention was identified in a human embryonic brain cDNA library. Preferably, it comprises a full-length polynucleotide sequence of 1284 bp, whose ORF (224-685) encodes 153 amino acids. Based on amino acid homology comparison, it is found that the encoded polypeptide is 58% homologous to human zinc finger protein FPM315. This novel human human zinc finger protein FPM315-17 has similar structures and biological functions to those of the human zinc finger protein FPM315.

The polynucleotide according to the invention may be in the forms of DNA or RNA. The forms of DNA include cDNA, genomic DNA, and synthetic DNA, etc., in single stranded or double stranded form. DNA may be an encoding strand or non-encoding strand. The coding sequence for mature polypeptide may be identical to the coding sequence shown in SEQ ID NO: 1, or is a degenerate sequence. As used herein, the term “degenerate sequence” means an sequence which encodes a protein or peptide comprising a sequence of SEQ ID NO: 2 and which has a nucleotide sequence different from the sequence of coding region in SEQ ID NO: 1.

The polynucleotide encoding the mature polypeptide of SEQ ID NO: 2 includes those encoding only the mature polypeptide, those encoding mature polypeptide plus various additional coding sequence, the coding sequence for mature polypeptide (and optional additional encoding sequence) plus the non-coding sequence.

The term “polynucleotide encoding the polypeptide” includes polynucleotides encoding said polypeptide and polynucleotides comprising additional coding and/or non-coding sequences.

The invention further relates to variants of the above polynucleotides which encode a polypeptide having the same amino acid sequence of invention, or a fragment, analogue and derivative of said polypeptide. The variant of the polynucleotide may be a naturally occurring allelic variant or a non-naturally occurring variant. Such nucleotide variants include substitution, deletion, and insertion variants. As known in the art, an allelic variant may have a substitution, deletion, and insertion of one or more nucleotides without substantially changing the functions of the encoded polypeptide.

The present invention further relates to polynucleotides which hybridize to the hereinabove-described sequences. That is, there is at least 50% and preferably at least 70% identity between the sequences. The present invention particularly relates to polynucleotides, which hybridize to the polynucleotides of the invention under stringent conditions. As herein used, the term “stringent conditions” means the following conditions: (1) hybridization and washing under low ionic strength and high temperature, such as 0.2×SSC, 0.1% SDS, 60° C.; or (2) hybridization after adding denaturants, such as 50% (v/v) formamide, 0.1% bovine serum/0.1% Ficoll, 42° C.; or (3) hybridization only when the homology of two sequences at least 95%, preferably 97%. Further, the polynucleotides which hybridize to the hereinabove described polynucleotides encode a polypeptide which retains the same biological function and activity as the mature polypeptide of SEQ ID NO: 2

The invention also relates to nucleic acid fragments hybridized with the hereinabove sequence. As used in the present invention, the length of the “nucleic acid fragment” is at least more than 10 bp, preferably at least 20-30 bp, more preferably at least 50-60 bp, and most preferably at least 100 bp. The nucleic acid fragment can be used in amplification techniques of nucleic acid, such as PCR, so as to determine and/or isolate the polynucleotide encoding human zinc finger protein FPM315-17.

The polypeptide and polynucleotide of the invention are preferably in the isolated form, preferably purified.

According to the invention, the specific nucleic acid sequence encoding human zinc finger protein FPM315-17 can be obtained in various ways. For example, the polynucleotide is isolated by hybridization techniques well-known in the art, which include, but are not limited to 1) the hybridization between a probe and genomic or cDNA library so as to select a homologous polynucleotide sequence, and 2) antibody screening of expression library so as to obtain polynucleotide fragments encoding polypeptides having common structural features.

According to the invention, DNA fragment sequences may further be obtained by the following methods: 1) isolating double-stranded DNA sequence from genomic DNA; and 2) chemical synthesis of DNA sequence so as to obtain the double-stranded DNA.

Among the above methods, the isolation of genomic DNA is the least frequently used. A commonly used method is the direct chemical synthesis of DNA sequence. A more frequently used method is the isolation of cDNA sequence. Standard methods for isolating the cDNA of interest is to isolate mRNA from donor cells that highly express said gene followed by reverse transcription of mRNA to form plasmid or phage cDNA library. There are many established techniques for extracting mRNA and the kits are commercially available (e.g. Qiagene). Conventional method can be used to construct cDNA library (Sambrook, et al., Molecular Cloning, A Laboratory Manual, Cold Spring Harbor Laboratory. New York, 1989). The cDNA libraries are also commercially available. For example, Clontech Ltd. has various cDNA libraries. When PCR is further used, even an extremely small amount of expression products can be cloned.

Numerous well-known methods can be used for screening for the polynucleotide of the invention from cDNA library. These methods include, but are not limited to, (1) DNA-DNA or DNA-RNA hybridization; (2) the appearance or loss of the function of a marker-gene; (3) the determination of the level of human zinc finger protein FPM315-17 give transcripts; (4) the determination of protein product of gene expression by immunology methods or biological activity assays. The above methods can be used alone or in combination.

In method (1), the probe used in the hybridization could be homologous to any portion of polynucleotide of invention. The length of probe is typically at least 10 nucleocides, preferably at least 30 nucleocides, more preferably at least 50 nucleocides, and most preferably at least 100 nucleotides. Furthermore, the length of the probe is usually less than 2000 nucleotides, preferably less than 1000 nucleotides. The probe usually is the DNA sequence chemically synthesized on the basis of the sequence information. Of course, the gene of the invention itself or its fragment can be used as a probe. The labels for DNA probe include, e.g., radioactive isotopes, fluoresceins or enzymes such as alkaline phosphatase.

In method (4), the detection of the protein products expressed by human zinc finger protein FPM315-17 gene can be carried out by immunology methods, such as Western blotting, radioimmunoassay, and ELISA.

The method of amplification of DNA/RNA by PCR (Saiki, et al. Science 1985; 230:1350-1354) is preferably used to obtain the polynucleotide of the invention. Especially when it is difficult to obtain the full-length cDNA, the method of RACE (RACE-cDNA terminate rapid amplification) is preferably used. The primers used in PCR can be selected according to the polynucleotide sequence information of the invention disclosed herein, and can be synthesized by conventional methods. The amplified DNA/RNA fragments can be isolated and purified by conventional methods such as gel electrophoresis.

Sequencing of polynucleotide sequence of the gene of the invention or its. various DNA fragments can be carried out by the conventional dideoxy sequencing method (Sanger et al. PNAS, 1977, 74: 5463-5467). Sequencing of polynucleotide sequence can also be carried out using the commercially available sequencing kits. In order to obtain the full-length cDNA sequence, it is necessary to repeat the sequencing process. Sometimes, it is needed to sequence the cDNA of several clones to obtain the full-length cDNA sequence.

The invention further relates to a vector comprising the polynucleotide of the invention, a genetically engineered host cell transformed with the vector of the invention or directly with the sequence encoding human zinc finger protein FPM315-17, and a method for producing the polypeptide of the invention by recombinant techniques.

In the present invention, the polynucleotide sequences encoding human zinc finger protein FPM315-17 may be inserted into a vector to form a recombinant vector containing the polynucleotide of the invention. The term “vector” refers to a bacterial plasmid, bacteriophage, yeast plasmid, plant virus or mammalian virus such as adenovirus, retrovirus or any other vehicle known in the art. Vectors suitable for use in the present invention include, but are not limited to the T7-based expression vector for expression in bacteria (Rosenberg, et al., Gene, 56:125, 1987), the PMSXND expression vector for expression in mammalian cells (Lee and Nathans, J Biol. Chem., 263:3521, 1988) and baculovirus-derived vectors for expression in insect cells. Any plasmid or vector can be used to construct the recombinant expression vector as long as it can replicate and is stable in the host. One important feature of an expression vector is that the expression vector typically contains an origin of replication, a promoter, a marker gene as well as translation regulatory components.

Methods known in the art can be used to construct an expression vector containing the DNA sequence of human zinc finger protein FPM315-17 and appropriate transcription/translation regulatory components. These methods include in vitro recombinant DNA technique, DNA synthesis technique, in vivo recombinant technique and so on (Sambroook, et al. Molecular Cloning, a Laboratory Manual, cold Spring Harbor Laboratory. New York, 1989). The DNA sequence is operatively linked to a proper promoter in an expression vector to direct the synthesis of mRNA. Exemplary promoters are lac or trp promoter of E.coli; PL promoter of A phage; eukaryotic promoters including CMV immediate early promoter, HSV thymidine kinase promoter, early and late SV40 promoter, LTRs of retrovirus, and other known promoters which control gene expression in the prokaryotic cells, eukaryotic cells or viruses. The expression vector may further comprise a ribosome binding site for initiating translation, transcription terminator and the like. Transcription in higher eukaryotes is increased by inserting an enhancer sequence into the vector. Enhancers are cis-acting elements of DNA, usually about from 10 to 300 bp in length that act on a promoter to increase gene transcription level. Examples include the SV40 enhancer on the late side of the replication origin 100 to 270 bp, the polyoma enhancer on the late side of the replication origin, and adenovirus enhancers.

Further, the expression vector preferably comprises one or more selective marker genes to provide a phenotype for the selection of the transformed host cells, e.g., the dehydrofolate reductase, neomycin resistance gene and GFP (green flurencent protein) for eukaryotic cells, as well as tetracycline or ampicillin resistance gene for E. coli.

The skilled in the art know clearly how to select appropriate vectors, transcriptional regulatory elements, e.g., promoters, enhancers, and selective marker genes.

According to the invention, polynucleotide encoding human zinc finger protein FPM315-17 or recombinant vector containing said polynucleotide can be transformed or transfected into host cells to construct genetically engineered host cells containing said polynucleotide or said recombinant vector. The term “host cell” means prokaryote, such as bacteria; or primary eukaryote, such as yeast; or higher eukaryotic, such as mammalian cells. Representative examples are bacterial cells, such as E. coli, Streptomyces, Salmonella typhimurium; fungal cells, such as yeast; plant cells; insect cells such as Drosophila S2 or Sf9; animal cells such as CHO, COS or Bowes melanoma.

Transformation of a host cell with the DNA sequence of the invention or a recombinant vector containing the DNA sequence may be carried out by conventional techniques as are well known to those ordinarily skilled in the art. When the host is prokaryotic, such as E. coli, competent cells, which are capable of DNA uptake, can be prepared from cells harvested at the exponential growth phase and subsequently treated by the CaCl2 method using procedures well known in the art. Alternatively, MgCl2 can be used. Transformation can also be carried out by electroporation, if desired. When the host is an eukaryote, transfection methods as well as calcium phosphate precipitation may be used. Conventional mechanical procedures such as micro-injection, electroporation, or liposome-mediated transfection may also be used.

The recombinant human zinc finger protein FPM315-17 can be expressed or produced by the conventional recombinant DNA technology (Science, 1984; 224:1431), using the polynucleotide sequence of the invention. The steps generally include:
(1) transfecting or transforming the appropriate host cells with the polynucleotide (or variant) encoding human zinc finger protein FPM315-17 of the invention or the recombinant expression vector containing said polynucleotide;
(2) culturing the host cells in an appropriate medium; and
(3) isolating or purifying the protein from the medium or cells.

In Step (2) above, depending on the host cells used, the medium for cultivation can be selected from various conventional mediums. The host cells are cultured under a condition suitable for its growth until the host cells grow to an appropriate cell density. Then, the selected promoter is induced by appropriate means (e.g., temperature shift or chemical induction) and cells are cultured for an additional period.

In Step (3), the recombinant polypeptide may be included in the cells, or expressed on the cell membrane, or secreted out of the cell. If desired, physical, chemical and other properties can be utilized in various isolation methods to isolate and purify the recombinant protein. These methods are well-known to those skilled in the art and include, but are not limited to conventional renaturation treatment, treatment by a protein precipitant (such as salt precipitation), centrifugation, cell lysis by osmosis, sonication, supercentrifugation, molecular sieve chromatography or gel chromatography, adsorption chromatography, ion exchange chromatagraphy, HPLC, and any other liquid chromatography, and a combination thereof.

### BRIEF DESCRIPTION OF THE DRAWINGS

The following drawings are provided to illustrate the embodiment of the invention, not to limit the scope of invention defined by the claims.

FIG. 1 shows an alignment comparison of amino acid sequences of human zinc finger protein FPM315-17 of the invention and human zinc finger protein FPM315. The upper sequence is human zinc finger protein FPM315-17, and the lower sequence is human zinc finger protein FPM315. The identical and similar amino acids are indicated by a one-letter code of amino acid, and “+,” respectively.

FIG. 2 shows the SDS-PAGE of the isolated human zinc finger protein FPM315-17, which has a molecular weight of 17kDa. The isolated protein band is marked with an arrow.

### EXAMPLES

The invention is further illustrated by the following examples. It is appreciated that these examples are only intended to illustrate the invention, not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

### Example 1

### Cloning of Human Zinc Finger Protein FPM315-17 Gene

Total RNA from a human embryonic brain was extracted by the one-step method with guanidinium isocyanate/phenol/chloroform. The poly(A) mRNA was isolated from the total RNA with Quik mRNA Isolation Kit (Qiegene). cDNA was prepared by reverse transcription with 2 μg poly(A) mRNA. The cDNA fragments were inserted into the polyclonal site of the pBSK(+) vector (Clontech) using Smart cDNA cloning kit (Clontech) and then transformed into DH5α cells to form the cDNA library. The 5′- and 3′-ends of all clones were sequenced with the Dye Terminate Cycle Reaction Sequencing Kit (Perkin-Elmer) and ABI 377 Automatic Sequencer (Perkin-Elmer). The sequenced cDNA were compared with the public database of DNA sequences (Genebank) and the DNA sequence of one clone 1268E10 was found to be a novel DNA sequence. The inserted cDNA sequence of clone 1268E10 was dual-directionally sequenced with a serial of synthesized primers. The full length cDNA contained in clone 1268E10 was 1284 bp (SEQ ID NO: 1) with a 462 bp ORF located in positions 224-685 which encodes a novel protein (SEQ ID NO: 2). This clone was named pBS-1268E10 and the encoded protein was named human zinc finger protein FPM315-17.

### Example 2

### Homology Search of cDNA Clone

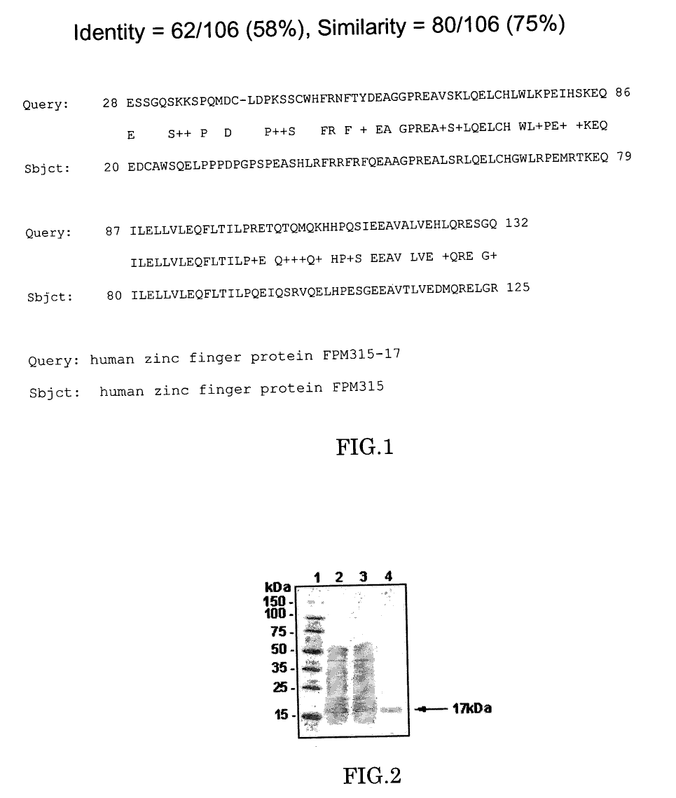

Homology search of the DNA sequence and its protein sequence of human zinc finger protein FPM315-17 of the invention were performed by BLAST (Basic Local Alignment Search Tool) (Altschul, S F et al. J. Mol. Biol., 1990; 215:403-10) in databases such as Genbank, Swissport, etc. The most homologous gene to human zinc finger protein FPM315-17 of the invention is the known human zinc finger protein FPM315. The Genbank accession number of its encoded protein is AC004232. The alignment result of the protein is shown in FIG. 1. The two proteins are highly homologous with a percent identity of 58% and a percent similarity of 75%.

### Example 3

### Cloning Human Zinc Finger Protein FPM315-17 Gene by RT-PCR

The template was total RNA extracted from a human embryonic brain. Reverse transcription was carried out with oligo-dT primer to produce cDNAs. After the cDNA was purified with a Qiagen Kit, PCR was carried out with the following primers:

(SEQ ID NO:3)

Primer 1: 5′-GGTGGGCTGGCGGTTGCGCTCCTC-3′

(SEQ ID NO:4)

Primer 2: 5′-CATAGGCCGAGGCGGCCCGACATG-3′

Primer 1 is the forward sequence starting from position 1 of 5′ end of SEQ ID NO: 1.

Primer 2 is the reverse sequence of the 3′ end of SEQ ID NO: 1.

The amplification condition was a 50 μl reaction system containing 50 mmol/L KCl, 10 mmol/L Tris-Cl (pH8.5), 1.5 mmol/L MgCl₂, 200 μmol/L dNTP, 10 pmol of each primer, 1U Taq DNA polymerase (Clontech). The reaction was performed on a PE 9600 DNA amplifier with the following parameters: 94° C. 30 sec, 55° C. 30 sec, and 72° C. 2 min for 25 cycles. β-actin was used as a positive control, and a blank template, as a negative control in RT-PCR. The amplified products were purified with a QIAGEN kit, and linked with a pCR vector (Invitrogen) using a TA Cloning Kit. DNA sequencing results show that the DNA sequence of PCR products was identical to nucleotides 1-1284 of SEQ ID NO: 1.

### Example 4

### Northern Blotting of Expression Product of Human Zinc Finger Protein FPM315-17 Gene

Total RNA was extracted by the one-step method (Anal. Biochem 1987, 162, 156-159) with guanidinium isocyanate-phenol-chloroform as follows. That is, homogenate the cells using 4M guanidinium isocyanate-25 mM sodium citrate, 0.2 sodium acetate (pH4.0), add 1 volume phenol and ⅕ volume chloroform-isoamyl alcohol (49:1), centrifuge after mixing. Take out the water phase, add 0.8 volume isopropyl alcohol, then centrifuge the mixture. Wash the RNA precipitation using 70% ethanol, then dry, then dissolve it in the water. 20 μg RNA was electrophoresed on the 1.2% agarose gel containing 20 mM 3-(N-morpholino)propane sulfonic acid (pH 7.0)-5 mM sodium acetate-1 mM EDTA-2.2M formaldehyde. Then transfer it to a nitrocellulose filter. Prepare the ³²P-labelled DNA probe with α-³²P DATP by random primer method. The DNA probe used is the coding sequence (224 bp-685 bp) of human zinc finger protein FPM315-17 amplified by PCR indicated in FIG. 1. The nitrocellulose filter with the transferred RNA was hybridized with the ³²P-labelled DNA probe (2×10⁶ cpm/ml) overnight in a buffer containing 50% formamide-25mM KH₂PO₄ (Ph7.4)-5× Denhardt's solution and 200 μg/ml salmine. Then wash the filter in the 1×SSC-0.1% SDS, at 55° C., for 30 min. Then analyze and quantify using a Phosphor Imager.

### Example 5

### In vitro Expression, Isolation and Purification of Recombinant Human Zinc Finger Protein FPM315-17

A pair of primers for specific amplification was designed based on SEQ ID NO: 1 and the encoding region in FIG. 1, the sequences are as follows:

(SEQ ID No:5)

Primer 3: 5′-CCCCATATGATGATGATGGTAGATCTGAAAGTG-3′

(SEQ ID No:6)

Primer 4: 5′-CATGGATCCCTATATCCACCTGCATCCTATTTC-3′

These two primers contain a NdeI and BamHI cleavage site on the 5′ end respectively. Within the sites are the coding sequences of the 5′ and 3′ end of the desired gene. NdeI and BamHI cleavage sites correspond to the selective cleavage sites on the expression vector pET-28b(+) (Novagen, Cat. No. 69865.3). PCR amplification was performed with the plasmid pBS-1268E10 containing the full-length target gene as a template. The PCR reaction was subject to a 50 μl system containing 10 pg pBS-1268E10 plasmid, 10 pmol of Primer-3 and 10 pmol of Primer-4, 1 μl of Advantage polymerase Mix (Clontech). The parameters of PCR were 94° C. 20 sec, 60° C. 30 sec, and 68° C. 2 min for 25 cycles. After digesting the amplification products and the plasmid pET-28(+) by NdeI and EcoRI, the large fragments were recovered and ligated with T4 ligase. The ligated product was transformed into E.coli DH5× cells with the calcium chloride method. After cultured overnight on a LB plate containing a final concentration of 30 μg/ml kanamycin, positive clones were selected using colony PCR and then sequenced. The positive clone (pET-1268E10) with the correct sequence was selected and the recombinant plasmid thereof was transformed into BL21(DE3)plySs (Novagen) using the calcium chloride method. In an LB liquid medium containing a final concentration of 30 μg/ml of kanamycin, the host bacteria BL21(pET-1268E10) were cultured at 37° C. to the exponential growth phase, then IPTG were added with the final concentration of 1 mmol/L, the cells were cultured for another 5 hours, and then centrifuged to harvest the bacteria. After the bacteria were sonicated, the supernatant was collected by centrifugation. Then the purified desired protein—human zinc finger protein FPM315-17 was obtained by a His.Bind Quick Cartridge (Novagen) affinity column with binding 6His-Tag. SDS-PAGE showed a single band at 17 kDa (FIG. 2). The band was transferred onto the PVDF membrane and the N terminal amino acid was sequenced by Edams Hydrolysis, which shows that the first 15 amino acids on N-terminus were identical to those in SEQ ID NO: 2.

### Example 6

### Preparation of Antibody Against Human Zinc Finger Protein FPM315-17

The following specific human zinc finger protein FPM315-17 polypeptide was synthesized by a polypeptide synthesizer (PE-ABI): NH2-Met-Met-Met-Val-Asp-Leu-Lys-Val-Ala-Ala-Tyr-Leu-Asp-Pro-Gln-COOH (SEQ ID NO:7). The polypeptide was conjugated with hemocyanin and bovine serum albumin (BSA) respectively to form two composites (See Avrameas et al., Immunochemistry,1969, 6:43). 4 mg of hemocyanin-polypeptide composite was used to immunize rabbit together with Freund's complete adjuvant. The rabbit was re-immunized with the hemocyanin-polypeptide composite and Freund's incomplete adjuvent 15 days later. The titer of antibody in the rabbit sera was determined with a titration plate coated with 15 μg/ml BSA-polypeptide composite by ELISA. The total IgG was isolated from the sera of an antibody positive rabbit with Protein A-Sepharose. The polypeptide was bound to Sepharose 4B column activated by cyanogen bromide. The antibodies against the polypeptide were isolated from the total IgG by affinity chromatography. The immunoprecipitation showed that the purified antibodies could specifically bind to human zinc finger protein FPM315-17.

### Example 7

### Application of the Polynucleotide Fragments as Hybrid Probes

Oligonucleotides selected from the polynucleotide of the instant invention can be versatilly applied as hybrid probes. The probes could be used to determine the existence of polynucleotide of the invention or its homologous polynucleotide sequences by hybridization with genomic, or cDNA libraries from normal or clinical tissues of various origins. The probes could be further used to determine whether polynucleotide of the invention or its homologous polynucleotide sequences are abnormally expressed in cells from normal or clinical tissues.

The aim of the following example is to select suitable oligonucletide fragments from SEQ ID NO: 1 as hybird probes to apply in membrane hybridization to determine whether there is polynucleotide of said invention or its homologous polynucleotide sequences in examined tissues. Membrane hybridization methods include dot hybridization, Southern blot, Northern blot, and replica hybridization. All these methods follow nearly the same steps after the polynucleotide samples are immobilized on membranes. These same steps are: membranes with samples immobilized on are prehybridized in hybrid buffer not containing probes to block nonspecific binding sites of the samples on membranes. Then prehybridization buffer is replaced by hybridization buffer containing labeled probes and incubation continues at the appropriate temperature so probes hybridize with the target nucleotides. Free probes are washed off by a series of washing steps after the hybridization step. A high-stringency washing condition (relatively low salt concentration and high temperature) is applied to reduce the hybridization background but retain highly specific signal. Two types of probes are selected for the example: the first type is oligonucleotides identical or annealed to SEQ ID NO: 1 the second type is oligonucleotides partially identical or partially annealed to SEQ ID NO: 1. Dot blot method is applied in the said example for immobilization of the samples on membrane. The strongest specific signal is produced by hybridization between first type probes and samples after relatively stringent membrane washing steps.

Selection of Probes

The principles below should be followed and some things should be taken into consideration for selection of oligonucleotide fragments from SEQ ID NO: 1 as hybrid probes:
1. The optimal length of probes should be between eighteen and fifty nucleotides.
2. GC content should be between 30% and 70%, since nonspecific hybridization increases when GC content is more than 70%.
3. There should be no complementary regions within the probes themselves.
4. Probes satisfying the requirements above could be initially selected for further computer-aided sequence analysis, which includes homology comparison between the initial selected probes and its source sequence region (SEQ ID NO: 1), and other known genomic sequences and their complements. Generally, probes should not be used when they share fifteen identical continuous base pairs, or 85% homology with a non-target region.
5. Whether said initial selected probes should be chosen for final application depends on further experimental confirmation.

The following two probes are selected and synthesized after the analysis above:

Probe one belongs to the first type probes, which is completely identical or annealed to the gene fragments of SEQ ID NO: 1(41 Nt);

(SEQ ID NO:8)

5′-TGATGATGGTAGATCTGAAAGTGGCTGCGTACTTGGACCCT-3′

Probe two belongs to the second type probes which is a replaced or mutant sequence of the gene fragments of SEQ ID NO: 1, or of its complementary fragments (41 Nt):

(SEQ ID NO:9)

5′-TGATGATGGTAGATCTGAAACTGGCTGCGTACTTGGACCCT-3′

Any other frequently used reagents unlisted but involved in the following concrete experimental steps and their preparation methods can be found in the reference: DNA PROBES G. H. Keller; M. M. Manak; Stockton Press, 1989 (USA) or a more commonly used molecular cloning experimental handbook (Molecular Cloning) (J. Sambrook et al. Acadimic press, 1998, 2nd edition)

Sample Preparation:

1) DNA Extraction from Fresh or Frozen Tissues

Steps: 1) move the fresh or newly thawed tissue (source tissue of the polyucleotide) onto a ice-incubated dish containing phosphate-buffered saline (PBS). Cut the tissue into small pieces with a scissor or an operating knife. Tissue should be kept moist through the operation. 2) mince the tissue by centrifugation at 2,000 g for 10 minutes. 3) resuspend the pellet (about 10 ml/g) with cold homogenating buffer (0.25 mol/i saccharose; 25 mmol/l Tris-HCl, pH 7.5; 25 m mol/L NaCl; 25 mmol/L MgCl2) at 4° C., and homogenate tissue suspension at full speed with an electronic homogenizer until it's completely smashed. 5) centrifuge at 1,000 g for 10 minutes. 6) resuspend the cell pellet (1-5 ml per 0.1 g initial tissue sample), and centrifuge at 1,000 g for 10 minutes. 7) resuspend the pellet with lysis buffer (1-5 ml per 0.1 g initial tissue sample), and continue to the phenol extraction method.

2) Phenol Extraction of DNA

Steps: 1) wash cells with 1-10 ml cold PBS buffer and centrifuge at 1000 g for 10 minutes. 2) resuspend the precipitated cells with at least 100 μl cold cell lysis buffer (1×10⁸ cells/ml). 3) add SDS to a final concentration of 1%. Addition of SDS into the cell precipitation before cell resuspension will cause the formation of large cell aggregates which are difficult to break and total yield will be reduced. This phenomenon is especially severe when extracting more than 107 cells. 5) incubate at 50° C. for an hour or shake gently overnight at 37° C. 6) add an equal volume of phenol: chloroform: isoamyl alcohol (25:24:1) to the DNA solution to be purified in a microcentrifuge tube, and centrifuge for 10 minutes. If the two phases are not clearly separated, the solution should be recentrifuged. 7) remove the water phase to a new tube. 8) add an equal volume of chloroform: isoamyl alcohol (24 : 1) and centrifuge for 10 minutes. 9) remove the water phase containing DNA to a new tube and then purify DNA by ethanol precipitation.

3) DNA Purification by Ethanol Precipitation

Steps: 1) add 1/10 vol of 2 mol/L sodium acetate and 2 vol of cold 100% ethanol into the DNA solution, mix and place at −20° C. for an hour or overnight. 2) centrifuge for 10 minutes. 3) carefully spill the ethanol. 4) add 500 μl of cold 70% ethanol to wash the pellet and centrifuge for 5 minutes. 6) carefully remove the ethanol and invert the tube on bibulous paper to remove the remaining ethanol. Air dry for 10-15 minutes to evaporate ethanol on pellet surface. But notice not to dry the pellet completely since completely dry pellet is difficult to be dissolved again. 7) resuspend the DNA pellet with a small volume of TE or water. Spin at low speed or blow with a drip tube, and add TE gradually and mix until DNA is completely dissolved. Add about 1 μl TE for every 1-5×10⁶ cells.

The following 8-13 steps are applied only when contamination must be removed, otherwise go to step 14 directly. 8) add RNase A into DNA solution to a final concentration of 100 μg/ml and incubate at 37° C. for 30 minutes. 9) add SDS and protease K to the final concentration of 0.5% and 100 μg/ml respectively, and incubate at 37° C. for 30 minutes. 10) add an equal volume of phenol: chloroform: isoamyl alcohol (25:24:1), and centrifuge for 10 minutes. 11) carefully remove the water phase and extract it with an equal volume of chloroform: isoamyl alcohol (24:1) and centrifuge for 10 minutes. 12) carefully remove the water phase, and add 1/10 vol of 2mol/L sodium acetate and 2.5 vol of cold 100% ethanol, then mix and place at −20° C. for an hour. 13) wash the pellet with 70% ethanol and 100% ethanol, air dry and resuspend DNA as same as steps 3-6. 14) determine the purity and production of DNA by A₂₆₀ and A₂₈₀ assay. 15) separate DNA sample into several portions and store at −20° C.

Preparation of Sample Membrane:

1) Take 4×2 pieces of nitrocellulose membrane (NC membrane) of desired size, and lightly mark out the sample dot sites and sample number with a pencil. Every probe needs two pieces of NC membrane, so then membranes could be washed under high stringency condition and stringency condition individually in the following experimental steps.

2) Pipette 15 μl of samples and control individually, dot them on the membrane, and dry at room temperature.

3) Place the membranes on filter paper soaked in 0.1 mol/l NaOH, 1.5 mol/L NaCl, leave for 5 minutes (twice), and allow to dry. Transfer the membranes on filter paper soaked in 0.5 mol/L Tris-HCl (pH7.0), 3 mol/L NaCl, leave for 5 minutes (twice), and allow to dry.

4) Place the membranes between clean filter paper, packet with aluminum foil, and vacuum dry at 60-80° C. for 2 hours.

Labeling of Probes

1) Add 3 μl probe (0.1 OD/10 μl), 2 μl kinase buffer, 8-10 μCi γ-³²P-dATP+2U Kinase, and add water to the final volume of 20 μl.

2) Incubate at 37° C. for 2 hours.

3) Add ⅕ vol bromophenol blue indicator (BPB).

4) Load that sample on Sephadex G-50 column.

5) Collect the first peak before the elution of ³²P-Probe (monitor the eluting process by Monitor).

6) Five drops each tube and collect for 10-15 tubes.

7) Measure the isotope amount with liquid scintillator

8) Merged collection of the first peak is the prepared ³²P-Probe (the second peak is free γ-³²P-dATP)

Prehybridization

Place the sample membranes in a plastic bag, add 3-10 mg prehybrid buffer (10× Denhardt's; 6×SSC, 0.1 mg/ml CT DNA (calf thymus gland DNA)), seal the bag, and shake on a 68° C. water bath for two hours hybridization.

Hybridization

Cut off a corner of the plastic bag, add in prepared probes, seal the bag, and shake on a 42° C. water bath overnight.

Membrane Washing Under a High-Stringency Condition:

1) Take out the hybridized sample membranes

2) Wash the membranes with 2×SSC, 0.1% SDS at 40° C. for 15 minutes (twice).

3) Wash the membranes with 0.1×SSC, 0.1% SDS at 40° C. for 15 minutes (twice).

4) Wash the membranes with 0.1×SSC, 0.1% SDS at 55° C. for 30 minutes (twice), and dry at room temperature.

Membrane Washing Under a Low-Stringency Condition:

1) Take out the hybridized sample membranes.

2) Wash the membranes with 2×SSC, 0.1% SDS at 37° C. for 15 minutes (twice).

3) Wash the membranes with 0.1×SSC, 0.1% SDS at 37° C. for 15 minutes (twice).

4) Wash the membranes with 0.1×SSC, 0.1% SDS at 40° C. for 15 minutes (twice), and dry at room temperature.

X Ray Autoradiography:

X ray autoradiograph at −70° C. (autoradiograph time varies according to radioactivity of the hybrid spots)

Experimental Results:

In hybridization experiments carried out under low-stringency membrane washing condition, the radioactivity of the above two probes hybridization spots show no obvious difference; while in hybridization experiments carried out under high-stringency membrane washing condition, radioactivity of the hybrid spot by probe one is obviously stronger than the other three's. So probe one could be applied in qualitative and quantitive analysis of the existence and differential expression of said invented polynucleotide in different tissues.

Industrial Applicability

The polypeptide of the invention and antagonists, agonists and inhibitors thereof can be directly used for the treatment of diseases, e.g., various malignant tumors or cancers, dermatitis, inflammation, adrenoprival disease and HIV infection and immune system diseases.

The zinc finger proteins generally participate in the expression and regulation of genes as a transcription factor and a signal transduction element. It is expressed in many tissues, such as hematopoietic cells, brain, nervous system, tissues related to tumors, and immortalized cells. The C2H2 type zinc finger structure containing proteins not only act as a important regulatory factor to the gene expression in many tissues, but also play key roles in the regulation of development. The KRAB containing Kruppel zinc finger proteins constitute a sub-family. The KRAB domain is related to the proper localization and function of these proteins.

It has been found that some proteins with C2H2 type zinc finger domain are related to the following diseases: Solid carcinomas such as tumor of thyroid, hysteromyoma; nervous system diseases, such as extrapyramidal system disfunction, Parkinson's disease, ataxia, nerve-cell tumour, glial cell tumor; blood malignancy diseases such as leucocythemia, non-Hodgkin's lymphoma(s). Development disorders such as Williams syndrome, cleft hand, cleft foot, Bechwith-Wiedemann syndrome; other tumors such as neurocytoma, colon cancer, mammary cancer and so on.

Zinc finger protein FPM315 is expressed in many tissues of infant and adult, including human tumor cell lines and lymphocyte cell lines etc. It is very important in many basic biological activities like the synthesis of the proteins and the metabolism of the cells.

The polypeptide of this invention and human zinc finger protein FPM315 are members of the human Kruppel zinc finger protein sub-family. They have similar physiological functions. It has been found that the new human zinc finger protein FPM315-17 participates in transcriptional regulation. It is highly expressed in tumor cell lines and lymphocyte cell lines. The abnormal expression of this protein will cause some related diseases.

Thus, the abnormal expression of the human zinc finger protein FPM315-17 in this invention will cause many diseases, especially tumors, neurological diseases, vicious blood dyscrasia, and development disorder. These include (but are not limited to):

Tumor of various tissues: tumor of thyroid, hysteromyoma, neuroblastoma, ependymocytoma, colon cancer, mammary cancer, leukemia, lymphoma, malignant histiocytosis, melanoma, sarcoma, carcinoma of stomach, liver cancer, lung cancer, esophagus cancer, bone marrow cancer, teratoma, adrenal gland cancer, carcinoma of the bladder, cancer of bone, osteosarcoma, brain cancer, uterus cancer, carcinoma of gallbladder, liver cancer, thymus tumour, astrocytoma, ependymoma, glial cell neoplasia neurofibroma, bone marrow cancer, endometrium cancer, nasal cavity tumour and Nasal sinus tumour, nasopharynx cancer, larynx cancer, tracheal tumors, fibroma, fibrosarcoma, adipoma, liposarcoma, leiomyoma.

Diseases of developmental disorder, such as extrapyramidal system dysfuncation, teratosis, Williams syndrome, Alagille syndrome, cleft hand, cleft foot, Bechwith-Wiedemann syndrome, congenital abortion, cleft palate, deficiency of limb,limb differentiation disorder, hyaline membrane disease, atelectasis, polycystic kidney, cryptorchidism, congenital inguinal hernia, uterus duplex, atresia of vagina, hypospadias, hermaphrodism, atrial septal defect, ventricular septal defect, pulmonary artery stenosis, patent ductus arteriosus, neural tube defect, congenital hydrocephalus, coloboma iridis, congenital glaucoma, congenital cataract, congenital deafness.

Nervous system diseases: Parkinson's disease, ataxia, nerve-cell tumour, glial cell tumor, senile dementia, depressive disorder, amnesia, Huntington disease, epilepsy, hemicrania, dementia, multiple sclerosis, myasthenia gravis, pseudohypertrophy, myotonic dystrophy, myodystony, schizophrenia, paranoia, anxiety neurosis, obsessive-compulsive neurosi, phobia, and nerves decline.

Blood malignancy diseases: leucocythemia, and non-Hodgkin's lymphoma(s) The abnormal expression of the human zinc finger protein FPM315-17 of the instant invention will cause some hereditary disease, endorcine system diseases and disorders such as endocrine adenomatosis, immune system diseases.

The polypeptide in this invention and its antagonist, activator and inhibitor can be directly used for treatment of diseases, such as tumors, neurological disease, blood diseases, developmental disorders, some genetic diseases, endorcine system diseases and disorders like endocrine adenomatosis, immune system diseases, etc.

The invention also provides methods for screening compounds so as to identify an agent which enhances human zinc finger protein FPM315-17 activity (agonists) or decrease human zinc finger protein FPM315-17 activity (antagonists). The agonists enhance the biological functions of human zinc finger protein FPM315-17 such as inactivation of cell proliferation, while the antagonists prevent and cure the disorders associated with the excess cell proliferation, such as various cancers. For example, in the presence of an agent, the mammal cells or the membrane preparation expressing human zinc finger protein FPM315-17 can be incubated with the labeled human zinc finger protein FPM315-17 to determine the ability of the agent to enhance or repress the interaction.

Antagonists of human zinc finger protein FPM315-17 include antibodies, compounds, receptor deletants and analogues. The antagonists of human zinc finger protein FPM315-17 can bind to human zinc finger protein FPM315-17 and eliminate or reduce its function, or inhibit the production of human zinc finger protein FPM315-17, or bind to the active site of said polypeptide so that the polypeptide can not function biologically.

When screening for compounds as an antagonist, human zinc finger protein FPM315-17 may be added into a biological assay. It can be determined whether the compound is an antagonist or not by determining its effect on the interaction between human zinc finger protein FPM315-17 and its receptor. Using the same method as that for screening compounds, receptor deletants and analogues acting as antagonists can be selected. Polypeptide molecules capable of binding to human zinc finger protein FPM315-17 can be obtained by screening a polypeptide library comprising various combinations of amino acids bound onto a solid matrix. Usually, human zinc finger protein FPM315-17 is labeled in the screening.

The invention further provides a method for producing antibodies using the polypeptide, and its fragment, derivative, analogue or cells as an antigen. These antibodies may be polyclonal or monoclonal antibodies. The invention also provides antibodies against epitopes of human zinc finger protein FPM315-17. These antibodies include, but are not limited to, polyclonal antibody, monoclonal antibody, chimeric antibody, single-chain antibody, Fab fragment and the fragments produced by a Fab expression library.

Polyclonal antibodies can be prepared by immunizing animals, such as rabbit, mouse, and rat, with human zinc finger protein FPM315-17. Various adjuvants, including but are not limited to Freund's adjuvant, can be used to enhance the immunization. The techniques for producing human zinc finger protein FPM315-17 monoclonal antibodies include, but are not limited to, the hybridoma technique (Kohler and Milstein. Nature,1975, 256:495-497), the trioma technique, the human B-cell hybridoma technique, the EBV-hybridoma technique and so on. A chimeric antibody comprising a constant region of human origin and a variable region of non-human origin can be produced using methods well-known in the art (Morrison et al, PNAS,1985,81:6851). Furthermore, techniques for producing a single-chain antibody (U.S. Pat. No. 4946778) are also useful for preparing single-chain antibodies against human zinc finger protein FPM315-17.

The antibody against human zinc finger protein FPM315-17 can be used in immunohistochemical method to detect the presence of human zinc finger protein FPM315-17 in a biopsy specimen.

The monoclonal antibody specific to human zinc finger protein FPM315-17 can be labeled by radioactive isotopes, and injected into human body to trace the location and distribution of human zinc finger protein FPM315-17. This radioactively labeled antibody can be used in the non-wounding diagnostic method for the determination of tumor location and metastasis.

Antibodies can also be designed as an immunotoxin targeting a particular site in the body. For example, a monoclonal antibody having high affinity to human zinc finger protein FPM315-17 can be covalently bound to bacterial or plant toxins, such as diphtheria toxin, ricin, ormosine. One common method is to challenge the amino group on the antibody with sulfydryl cross-linking agents, such as SPDP, and bind the toxin onto the antibody by interchanging the disulfide bonds. This hybrid antibody can be used to kill human zinc finger protein FPM315-17-positive cells.

The antibody of the invention is useful for the therapy or the prophylaxis of disorders related to the human zinc finger protein FPM315-17. The appropriate amount of antibody can be administrated to stimulate or block the production or activity of human zinc finger protein FPM315-17.

The invention further provides diagnostic assays for quantitative and in situ measurement of human zinc finger protein FPM315-17 level. These assays are well known in the art and include FISH assay and radioimmunoassay. The level of human zinc finger protein FPM315-17 detected in the assay can be used to illustrate the importance of human zinc finger protein FPM315-17 in diseases and to determine the diseases associated with human zinc finger protein FPM315-17.

The polypeptide of the invention is useful in the analysis of polypeptide profile. For example, the polypeptide can be specifically digested by physical, chemical, or enzymatic means, and then analyzed by one, two or three dimensional gel electrophoresis, preferably by spectrometry.

New human zinc finger protein FPM315-17 polynucleotides also have many therapeutic applications. Gene therapy technology can be used in the therapy of abnormal cell proliferation, development or metabolism, which are caused by the loss of human zinc finger protein FPM315-17 expression or the abnormal or non-active expression of human zinc finger protein FPM315-17. Recombinant gene therapy vectors, such as virus vectors, can be designed to express mutated human zinc finger protein FPM315-17 so as to inhibit the activity of endogenous human zinc finger protein FPM315-17. For example, one form of mutated human zinc finger protein FPM315-17 is a truncated human zinc finger protein FPM315-17 whose signal transduction domain is deleted. Therefore, this mutated human zinc finger protein FPM315-17 can bind the downstream substrate without the activity of signal transduction. Thus, the recombinant gene therapy vectors can be used to cure diseases caused by abnormal expression or activity of human zinc finger protein FPM315-17. The expression vectors derived from a virus, such as retrovirus, adenovirus, adeno-associated virus, herpes simplex virus, parvovirus, and so on, can be used to introduce the human zinc finger protein FPM315-17 gene into the cells. The methods for constructing a recombinant virus vector harboring human zinc finger protein FPM315-17 gene are described in the literature (Sambrook, et al. supra). In addition, the recombinant human zinc finger protein FPM315-17 gene can be packed into liposome and then transferred into the cells.

The methods for introducing the polynucleotides into tissues or cells include directly injecting the polynucleotides into tissue in the body; or introducing the polynucleotides into cells in vitro with vectors, such as virus, phage, or plasmid, etc, and then transplanting the cells into the body.

Also included in the invention are ribozymes and oligonucleotides, including antisense RNA and DNA, which inhibit the translation of the human zinc finger protein FPM315-17 mRNA. Ribozyme is an enzyme-like RNA molecule capable of specifically cutting certain RNA. The mechanism is nucleic acid endo-cleavage following specific hybridization of ribozyme molecule and the complementary target RNA. Antisense RNA and DNA as well as ribozyme can be prepared by using any conventional techniques for RNA and DNA synthesis, e.g., the widely used solid phase phosphite chemical method for oligonucleotide synthesis. Antisense RNA molecule can be obtained by the in vivo or in vitro transcription of the DNA sequence encoding said RNA, wherein said DNA sequence is integrated into the vector and downstream of the RNA polymerase promoter. In order to increase its stability, a nucleic acid molecule can be modified in many manners, e.g., increasing the length of two the flanking sequences, replacing the phosphodiester bond with the phosphothioester bond in the oligonucleotide.

The polynucleotide encoding human zinc finger protein FPM315-17 can be used in the diagnosis of human zinc finger protein FPM315-17 related diseases. The polynucleotide encoding human zinc finger protein FPM315-17 can be used to detect whether human zinc finger protein FPM315-17 is expressed or not, and whether the expression of human zinc finger protein FPM315-17 is normal or abnormal in the case of diseases. For example, human zinc finger protein FPM315-17 DNA sequences can be used in the hybridization with biopsy samples to determine the expression of human zinc finger protein FPM315-17. The hybridization methods include Southern blotting, Northern blotting and in situ blotting, etc., which are well-known and established techniques. The corresponding kits are commercially available. A part of or all of the polynucleotides of the invention can be used as probe and fixed on a microarray or DNA chip for analysis of differential expression of genes in tissues and for the diagnosis of genes. The human zinc finger protein FPM315-17 specific primers can be used in RNA-polymerase chain reaction and in vitro amplification to detect transcripts of human zinc finger protein FPM315-17.

Further, detection of mutations in human zinc finger protein FPM315-17 gene is useful for the diagnosis of human zinc finger protein FPM315-17 -related diseases. Mutations of human zinc finger protein FPM315-17 include site mutation, translocation, deletion, rearrangement and any other mutations compared with the wild-type human zinc finger protein FPM315-17 DNA sequence. The conventional methods, such as Southern blotting, DNA sequencing, PCR and in situ blotting, can be used to detect a mutation. Moreover, mutations sometimes affects the expression of protein. Therefore, Northern blotting and Western blotting can be used to indirectly determine whether the gene is mutated or not.

Sequences of the present invention are also valuable for chromosome identification. The sequence is specifically targeted to and can hybridize with a particular location on an individual human chromosome. There is a current need for identifying particular sites of gene on the chromosome. Few chromosome marking reagents based on actual sequence data (repeat polymorphism) are presently available for marking chromosomal location. The mapping of DNA to chromosomes according to the present invention is an important first step in correlating those sequences with genes associated with disease.

Briefly, sequences can be mapped to chromosomes by preparing PCR primers (preferably 15-35 bp) from the cDNA. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the oligonucleotide primers of the invention, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clones to a metaphase chromosomal spread can be used to provide a precise chromosomal location in one step. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis.

Next, it is necessary to determine the differences in the cDNA or genomic sequence between affected and unaffected individuals. If a mutation is observed in some or all of the affected individuals but not in any normal individuals, then the mutation is likely to be the cause of the disease. Comparison of affected and unaffected individuals generally involves first looking for structural alterations in the chromosomes, such as deletions or translocations, that are visible from chromosome level, or detectable using PCR based on that DNA sequence. With current resolution of physical mapping and genetic mapping techniques, a cDNA precisely localized to a chromosomal region associated with the disease could be one of 50 to 500 potential causative genes. (This assumes 1 megabase mapping resolution and one gene per 20 kb).

According to the invention, the polypeptides, polynucleotides and its mimetics, agonists, antagonists and inhibitors may be employed in combination with a suitable pharmaceutical carrier. Such a carrier includes but is not limited to water, glucose, ethanol, salt, buffer, glycerol, and combinations thereof. Such compositions comprise a safe and effective amount of the polypeptide or antagonist, as well as a pharmaceutically acceptable carrier or excipient with no influence on the effect of the drug. These compositions can be used as drugs in disease treatment.

The invention also provides a pharmaceutical pack or kit comprising one or more containers filled with one or more of the ingredients of the pharmaceutical compositions of the invention. With such container(s) there may be a notice from a governmental agency, that regulates the manufacture, use or sale of pharmaceuticals or biological products, the notice reflects government's approval for the manufacture, use or sale for human administration. In addition, the polypeptides of the invention may be employed in conjunction with other therapeutic compounds.

The pharmaceutical compositions may be administered in a convenient manner, such as through topical, intravenous, intraperitoneal, intramuscular, subcutaneous, intranasal or intradermal routes. human zinc finger protein FPM315-i7 is administered in an amount, which is effective for treating and/or prophylaxis of the specific indication. The amount of human zinc finger protein FPM315-17 administrated on patient will depend upon various factors, such as delivery methods, the subject health, the judgment of the skilled clinician.

1-18. Canceled.

19. An isolated polypeptide having human zinc finger protein FPM315 activity and comprising an amino acid sequence of SEQ ID NO: 2, or its active fragments, analogues or derivatives.

20. An isolated polypeptide having a human zinc finger protein FPM315 activity and comprising an amino acid sequence that is at least 95% identical to SEQ ID NO: 2.

21. The polypeptide of claim 20 wherein said polypeptide comprises the amino acid sequence of SEQ ID NO: 2.

22. An isolated polynucleotide selected from the group consisting of:
(a) a polynucleotide encoding a polypeptide that has human zinc finger protein FPM315 activity and is at least 95% identical to SEQ ID NO: 2; or
(b) a polynucleotide complementary to polynucleotide (a).

23. A polynucleotide of claim 22 wherein the polynucleotide encodes an amino acid sequence of SEQ ID NO:2.

24. A polynucleotide of claim 22 wherein the sequence of said polynucleotide comprises position 224-685 of SEQ ID NO:1.

25. A polynucleotide of claim 24 wherein the sequence of said polynucleotide comprises position 1-1284 of SEQ ID NO:1.

26. A recombinant vector comprising a polynucleotide of claim 22, and a suitable regulatory element.

27. A genetically engineered host cell comprising a polynucleotide of claim 22.

28. A method for producing a polypeptide having an activity of a human zinc finger protein FPM315 which comprises the steps of:
(a) culturing an engineered host cell of claim 27 under conditions suitable for the expression of a human zinc finger protein FPM315; and
(b) isolating a polypeptides having the activity of a human zinc finger protein FPM315 from the culture.

29. An antibody which binds specifically for a polypeptide of claim 20.

30. A compound simulating, enhancing, antagonizing, or inhibiting a human zinc finger protein FPM315 activity of a polypeptide of claim 20.

31. A compound of claim 30 which is an antisense sequence of the polynucleotide sequence of SEQ ID NO: 1 or its fragment which inhibits expression of SEQ ID NO:1 in a cell.

32. A method for regulating the activity of a human zinc finger protein FPM315 in vivo or in vitro, comprising applying a compound of claim 30 to a human zinc finger protein FPM315 or a polynucleotide encoding human zinc finger protein FPM315.

33. A method for detecting a disease related to the polypeptide of claim 20, or for determining a susceptibility of a mammal thereto, said method comprising detecting the amount of expression of said polypeptide, or detecting the activity of said polypeptide, or detecting the nucleotide variant of a polynucleotide encoding the polypeptide which variant causes said abnormal expression or activity.

34. A method for screening mimetics, agonists, antagonists or inhibitors of a human zinc finger protein FPM315; or for peptide profiling, the method comprising labeling a polypeptide according to claim 20 and applying the labeled polypeptide in the screening or profiling.

35. A method for nucleic acid amplification, or for nucleic acid hybridization reaction, or for manufacture of gene chip or microarray, the method comprising synthesizing a polynucleotide of claim 22.

36. A pharmaceutical composition comprising a polypeptide according to claim 20, and a pharmaceutically acceptable carrier.

37. A method for the treatment of developmental disorders, diseases caused by abnormal metabolism of immune system, and cancers wherein the method comprising administering an effective amount of the pharmaceutical composition of claim 36 to a patient in need thereof.

38. A pharmaceutical composition comprising a polynucleotide according to claim 22, and a pharmaceutically acceptable carrier.


SEQUENCE LISTING (9 sequences)

SEQ ID NO: 1 - DNA, 1284 bp, Homo sapiens
  CDS (224)..(685)
ggtgggctggcggttgcgctcctcagatcggcggcctttcgggcggtggcttgcgtttgagcctcagaaagcgaggagcggcctccacggaagccaagctggccgagtgcttttaggaagaagatccttttattgcttttgtacaagaccagacaggatctcatttgttaaacgtggtaccaattgggtgtcttaacacaggagcagaacttcctagagcagaatgatgatggtagatctgaaagtggctgcgtacttggaccctcagatcagggctttgtgggagaccaaggggcctgcaagagagagctccggtcagagtaaaaaatctcctcaaatggactgtctcgatcctaagagctcttgctggcacttccggaatttcacctatgatgaagcaggtggaccccgtgaggctgtcagcaaacttcaagaattatgtcatctatggctgaagccagagatccactcaaaagagcagatactggaactgctggtgctggagcagttcctgactattctgcccagggagacacagacccagatgcagaagcaccatccacagagcattgaggaggctgtggctctggtagaacacttgcagagggaatctggtcaaacatggaatggggtgagaagaaagattcctgacatgtacagtgaaataggatgcaggtggatatagtagagatggtggcagttagttgagaaattagatggattaggtaggtcattgtttggtttttttttaaggacagttaaggtgggacttgaaactacaggcccacagaaaagttcacaggtttcagagattgtggtagatacttccctggccttagagaaagagagttgcttttgccctctttggagccagggtgaattgagggtgttgggcctagtctggcttggccaaggagacaggacttaagtacttagaaacagaataaaaggtaataaagagtcagggaggctggatgcagtggctcacacctgtaatctcagcactttaggaggctgaggcgggtagatcacctgaggtcaggagttcaagaccagcctggacagcatggtggaaccctctctctacaaaaataaacaaattagctgggcatgatgaccggtgcctgtaatcccggctatcctcgaggctgaggagggagaatcatttgaacctgggaggttgcagtgagccgagatcgcaccattgcactccagcctgggcaacacagcaagactccatcttcaaaaaaaaaaaaaaaacatgtcgggccgcctcggcctatg

SEQ ID NO: 2 - protein, 153 aa, Homo sapiens
MMMVDLKVAAYLDPQIRALWETKGPARESSGQSKKSPQMDCLDPKSSCWHFRNFTYDEAGGPREAVSKLQELCHLWLKPEIHSKEQILELLVLEQFLTILPRETQTQMQKHHPQSIEEAVALVEHLQRESGQTWNGVRRKIPDMYSEIGCRWI

SEQ ID NO: 3 - DNA, 24 bp, Artificial
  oligonucleotide primer
ggtgggctggcggttgcgctcctc

SEQ ID NO: 4 - DNA, 24 bp, Artificial
  oligonucleotide primer
cataggccgaggcggcccgacatg

SEQ ID NO: 5 - DNA, 33 bp, Artificial
  oligonucleotide primer
ccccatatgatgatgatggtagatctgaaagtg

SEQ ID NO: 6 - DNA, 33 bp, Artificial
  oligonucleotide primer
catggatccctatatccacctgcatcctatttc

SEQ ID NO: 7 - protein, 15 aa, Artificial
  Partial sequence of SEQ ID NO2
MMMVDLKVAAYLDPQ

SEQ ID NO: 8 - DNA, 41 bp, Artificial
  oligonucleotide primer
tgatgatggtagatctgaaagtggctgcgtacttggaccct

SEQ ID NO: 9 - DNA, 41 bp, Artificial
  oligonucleotide primer
tgatgatggtagatctgaaactggctgcgtacttggaccct